(19) United States
(12) Patent Application Publication
(10) Pub. No.: US 2015/0190163 A1
(43) Pub. Date: Jul. 9, 2015

(54) MEDICAL DEVICES FOR TISSUE EXTRACTION AND RELATED METHODS

(71) Applicant: Boston Scientific Scimed, Inc., Maple Grove, MN (US)

(72) Inventors: Ronald CIULLA, Westford, MA (US); Kenneth P. REEVER, Hopedale, MA (US)

(73) Assignee: Boston Scientific Scimed, Inc.

(21) Appl. No.: 14/547,967

(22) Filed: Nov. 19, 2014

Related U.S. Application Data
(60) Provisional application No. 61/923,880, filed on Jan. 6, 2014.

(51) Int. Cl. A61B 17/32

(52) U.S. Cl. CPC A61B 17/32002; A61B 2017/320024; A61B 2017/00561

(57) ABSTRACT

Embodiments of the present disclosure include medical devices, and related methods of use, and manufacture thereof. A medical device is disclosed having an inner member and an outer member. The inner member may have a sharp edge, and the outer member may have a window configured to receive tissue. The medical device may include a plurality of suction passageways, the plurality of suction passageways may extend through at least one or both the inner member and the outer member and may be located adjacent the window. In addition, the inner member may be configured to move relative to the outer member.

### CROSS REFERENCE TO RELATED APPLICATIONS

This application claims the benefit of U.S. Provisional Application No. 61/923,880, filed Jan. 6, 2014, the disclosure of which is incorporated herein by reference in its entirety.

### DESCRIPTION OF THE DISCLOSURE

1. Field of the Disclosure

Embodiments of the present disclosure relate generally to medical devices and procedures. In particular, embodiments of the present disclosure relate to medical devices and methods for tissue extraction/removal.

2. Background of the Disclosure

Benign Prostatic Hyperplasia (BPH) is noncancerous enlargement of the prostate gland in men. BPH includes hyperplasia (an increase in the number of cells) of prostatic stromal and epithelial cells which result in the formation of large nodules in the periurethral region of the prostate. As the prostate enlarges it puts pressure on and/or partially or completely occludes the urethra. Additionally, prostate enlargement may cause pain, difficulty in urination, infection, or the like.

Holmium Laser Enucleation of the Prostate (HoLEP) is a technique for treating BPH. HoLEP typically involves inserting a laser device into the urethra and directing the device to a target tissue including enlarged prostate tissue. Typically, such laser devices are directed to the target tissue using a sheath such as, for example, a cystoscope and/or rectoscope. The laser device enucleates (e.g., separates or removes) the target prostate tissue away from its surroundings. Typically the separated prostate tissue may form one or more large pieces of tissue, referred to as “tissue balls”, which are then directed (e.g., pushed) into the bladder using the laser device or another medical device. While referred to herein as a “ball,” the severed tissue may not necessarily be in the shape of a ball (e.g., sphere) but rather, may have any shape including irregular shapes. The laser device is then removed and another device such as a morcellator is introduced into the sheath for removing the tissue. A morcellator is a surgical device having a small opening at its distal end, one or more cutting blades, and suction capability. The blades may cut (e.g., mince, puree) the large pieces of tissue, e.g., tissue balls, that were moved into the bladder into smaller pieces. These smaller pieces may then be removed out of the body through the opening via, suction and/or other means.

Generally, tissue balls that are pushed into the bladder float freely within the bladder and have a tendency to avoid capture by bouncing off the bladder wall and/or floating away from the morcellator device, which makes it difficult to contact the tissue balls for morcellation. Some conventional devices and methods apply suction to draw the tissue balls closer to the morcellator/blades.

There are, however, disadvantages to using conventional devices and methods. For example, suction may be “choked” off during the operation by movement of the cutting blades. In a conventional device, suction may be applied initially to draw a portion of tissue into the device via an opening. In order to sever tissue, a cutting blade is moved so as to simultaneously cut through tissue drawn into the opening, and close the opening. Closing the opening, however, can “choke” (e.g., cut, stop, lessen, etc.) suction from being applied to remaining portions of the tissue ball (e.g., portions not initially received within the opening and severed by the cutting blade), which results in these portions being left to float freely in the bladder and bounce away from the conventional morcellator device. In such instances, a physician may need to reposition the conventional morcellator device within the bladder to draw the remaining tissue towards the device, which ultimately increases the time and effort required for the procedure. Accordingly, it may be desirable to provide for alternative systems and methods for tissue removal.

### SUMMARY OF THE DISCLOSURE

Embodiments of the present disclosure include a medical device, such as a tissue morcellation device, that may be used to remove excised tissue from a body and methods thereof.

In one exemplary embodiment, a medical device may include an inner member and an outer member. The inner member may be capable of resecting tissue. The outer member may have a window configured to receive tissue. The medical device may further include a plurality of suction passageways, the plurality of suction passageways may extend through at least one or both of the inner member and outer member and may be located adjacent the window.

The medical device may further include one or more of the following features: the inner member may include a sharp edge; the inner member and outer member may be concentrically arranged along a longitudinal axis; upon actuation, the inner member may be configured to move distally along the longitudinal axis between a proximal open position and a distal closed position; in the open position, the window may be configured to fluidly couple with the suction source, and in the closed position, the plurality of suction passageways may be configured to fluidly couple with the suction source; upon actuation, the inner member may be configured to rotate about the longitudinal axis between an open position and a closed position; in the open position, the window may be configured to fluidly couple with the suction source, and in the closed position, the plurality of suction passageways may be configured to fluidly couple with the suction source; a handle may be coupled to the inner and outer members and where the handle may be operably coupled to a source of vacuum pressure; and one or more actuators may be on the handle where the one or more actuators may be configured to control movement of the inner member relative to the outer member.

In another exemplary embodiment, a morcellator device is disclosed. The morcellator device may include an elongate working member. The working member may include a first member, a second member, and a plurality of suction passageways. The first member may be capable of resecting tissue. The second member may have a window extending through a sidewall thereof, wherein the first member may be configured to move relative to the second member. A plurality of suction passageways may extend through at least one or both of the first member and second member and may be located adjacent the window, wherein the plurality of suction passageways may be configured to fluidly couple to a suction source.

The morcellator device may further include one or more of the following features: the first member and second member may be concentrically arranged along a longitudinal axis; upon actuation, the first member may be configured to move distally along the longitudinal axis between a proximal open position and a distal closed position; in the open position, the window may be configured to fluidly couple with a source of suction, and wherein in the closed position, the plurality of suction passageways may be configured to fluidly couple with the suction source; the first member may have a sharpened edge; and in the open position, the window may be configured to fluidly couple with the suction source, and wherein in the closed position, the plurality of suction passageways may be configured to fluidly couple with the suction source.

In additional exemplary embodiment, a method of removing tissue from a body of a patient may include inserting a medical device proximate to a target tissue. The medical device may include a first member capable of resecting tissue and a second member having a window extending through a sidewall thereof. The first member may be configured to move relative to the second member. A plurality of suction passageways may extend through at least one or both of the first member and second member and may be posited adjacent the window. The method may include applying a source of suction to the medical device so as to draw target tissue into the window. The method may further include moving the first member relative to the second member so as to sever the target tissue. In addition, the method may include maintaining the suction through the first and second members via the plurality of suction passageways after moving the first member relative to the second member.

The method may further include one or more following features: the first member and second member may be concentrically arranged along a longitudinal axis; moving the first member relative to the second member may include moving the first member distally along the longitudinal axis between a proximal open position and a distal closed position; moving the first member relative to the second member may include rotating the first member about the longitudinal axis between an open position and a closed position; and in the open position, the window may be configured to fluidly couple with a source of suction, and wherein in the closed position, the plurality of suction passageways may be configured to fluidly couple with the source of suction.

Additional objects and advantages of the present disclosure will be set forth in part in the description which follows, and in part will be understood from the description, or may be learned by practice of the claimed disclosure. The objects and advantages of the claimed disclosure will be realized and attained by means of the elements and combinations particularly pointed out in the appended claims.

It is to be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 illustrates an exemplary conventional medical device for tissue removal; and

FIG. 2 illustrates a portion of an exemplary medical device, including a plurality of suction passageways, according to an embodiment of the present disclosure.

### DESCRIPTION OF THE EMBODIMENTS

Reference will now be made in detail to embodiments of the present disclosure, examples of which are illustrated in the accompanying drawings. Wherever possible, the same reference numbers will be used throughout the drawings to refer to the same or like parts. As used herein, the term “distal” refers to the direction that is away from the user and into the patient's body. By contrast, the term “proximal” refers to the direction that is closer to the user and away from the patient's body.

### Overview

Embodiments of the present disclosure may include a medical device for removing a material from a body of a patient and methods of use thereof. In some embodiments, the medical device may be used to remove tissue that has been cut away or otherwise severed from its surroundings. In at least one embodiment, the tissue to be removed may be tissue from the prostate for treatment of BPH. In alternative embodiments, the medical device may be used to remove other types of tissues or materials such as, for example, bladder stones, kidney stones, and the like. For convenience, the exemplary medical device discussed herein is referred to as an extraction device or a morcellator device; however, this reference is merely made for convenience, and is intended to include devices capable of other and/or additional operations and/or functions such as resection.

In the following sections, embodiments of the present disclosure will be described using the bladder as an exemplary body organ. It will be understood that the bladder is merely an example and that the device may be utilized in other parts of the body.

The present disclosure provides medical devices for removing tissue from a patient's body, such as, tissue extraction devices or morcellator devices. The medical devices may be used to remove tissue that has been cut away or excised from the body. An opening and a plurality of members of the medical device may facilitate removal of excised tissue from the body. The medical device may be configured to be introduced into the body through a suitable natural opening, such as through the urethra.

The devices and methods disclosed in the present disclosure can be used to treat the pelvic region of a male. The male pelvic region includes a urinary system having a urethra, a bladder, a prostate, a urinary meatus, and an ejaculatory duct. The urethra is a biological lumen connecting the bladder to the urinary meatus at the tip of the penis. The urethra connects to the bladder at a bladder opening. The prostate is positioned around the urethra between the bladder and the penis. Upon stimulation, the bladder constricts and urine is released out of the urinary meatus through the urethra.

In a male suffering from Benign Prostate Hyperplasia (BPH), however, the passage of urine through the urethra from the bladder to the urinary meatus is obstructed by an enlarged prostate. For example, excess tissue in the prostate may constrict (e.g., narrow, obstruct, and/or partially occlude) the urethra proximate to the bladder opening, and may cause pain, difficulty in urination, and/or urinary infections. For example, due to constriction of the urethra, urine is prevented from freely flowing through the urethra to exit the penis via the urinary meatus. In turn, it may result in urine buildup in the bladder. This accumulation of urine may increase infection and the occurrence of other urinary tract problems. In addition, the enlarged prostate may constrict (e.g., narrow, obstruct, and/or partially occlude) the ejaculatory duct causing erectile dysfunction or ejaculatory problems.

The enlarged prostate may be severed using various methods, such as, laser enucleation as is known in the art. In laser enucleation, a laser is used to cut (e.g., sever, ablate) tissue from remaining portions of the enlarged prostate. By way of example only, laser enucleation may be used to cut excess tissue region from the enlarged prostate in order to alleviate constriction of the urethra. Cutting or severing of such tissue may form a tissue ball. Once formed, the tissue ball may be moved (e.g., pushed) into the bladder for removal via an appropriate tool. The tissue ball is undesirable and may be removed using high-pulse energy to destroy the tissue ball, or by using a morcellator device to cut the tissue ball into small pieces. The present disclosure discloses a medical device that facilitates tissue removal in less time and with less effort by the physician as compared to the conventional devices and methods discussed above.

### Exemplary Embodiments

The present disclosure includes a device, for example, a morcellator device for removing tissue from a body. The device according to the present disclosure may be used to remove tissue that has been cut away or excised from the body, e.g., via a laser apparatus, blade, knife, or other devices, including devices using fluid, pressure, and/or temperature to separate tissue from the body. A plurality of passageways may be provided to facilitate appropriate and constant suction to the device such that a target tissue ball may be continuously drawn proximate to the morcellator device. The morcellator device may be configured to be introduced into the body through a suitable natural opening, such as through the urethra into the bladder to perform the desired function.

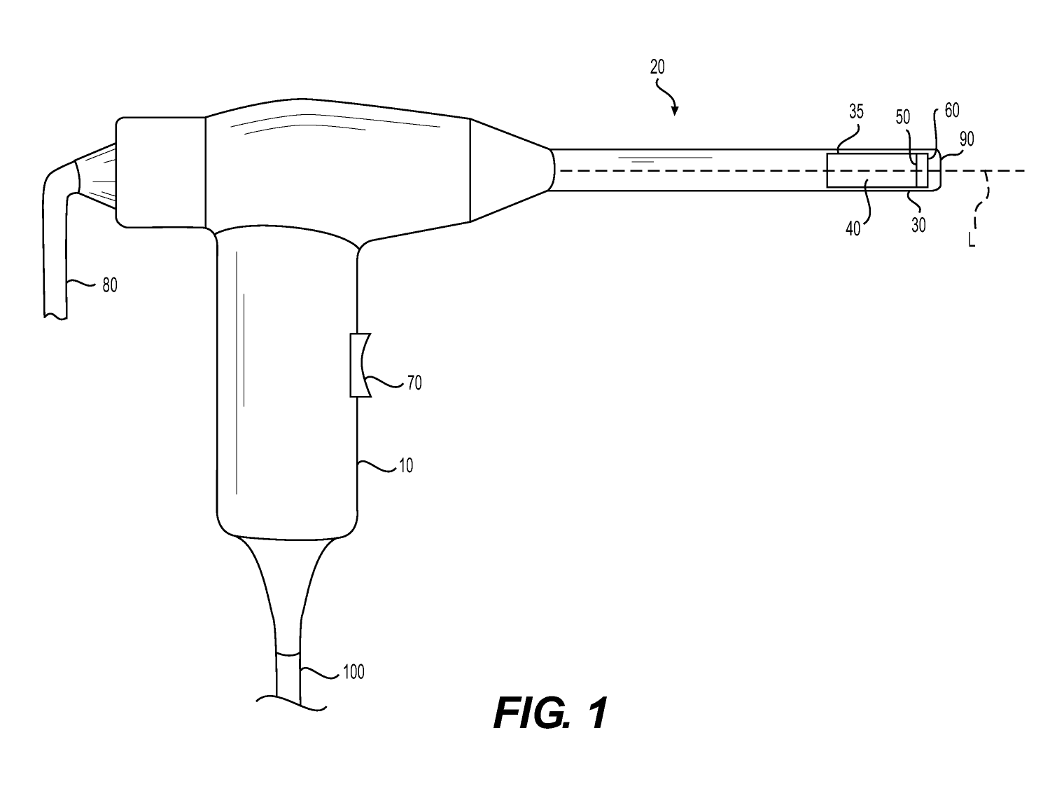

FIG. 1 shows an exemplary medical device employing an elongate working member 20. The medical device may be used to remove large pieces of severed tissue, e.g., tissue balls, from a patient's body. The elongate member 20 may be configured to be introduced into a patient's body through a suitable natural opening (e.g., the urethra) with the aid of a suitable introduction sheath (not shown).

The introduction sheath may include one or more working channels through which a physician may introduce one or more medical devices. For example, the introduction sheath may be a catheter, an endoscope, and/or any other appropriate luminal introducer structure configured to be inserted and manipulated within the patient's body. In some embodiments, the introduction sheath may be a cystoscope or a rectoscope that may be inserted into the urethra of a patient. The cystoscope, as is known in the art, is an endoscopic instrument that may be passed through the urethra for visualization and/or manipulation of tissue. In some embodiments, the introduction sheath may have a substantially circular cross-sectional shape. Other suitable cross-sectional shapes such as elliptical, oval, polygonal, or irregular may also be contemplated.

As shown, the elongate member 20 may include an outer member 30 (or a second member), and an inner member 40 (or a first member) disposed in the outer member 30. The outer member 30 may have a proximal end, distal end, and a longitudinal axis “L” extending between the proximal and distal ends. The outer member 30 may further include a sidewall defining a central lumen extending between the proximal end and the distal end. The sidewall may further define an opening 35 (e.g., window and/or passageway) configured to receive the target tissue. The lumen may be operably and selectively coupled to a suction source (e.g., vacuum pump not shown) via connection 80 to provide suction through the opening 35 to draw the tissue into the opening 35 and/or to remove tissue from the patient's body. Additionally, the outer member 30 may have a closed distal end 90 that helps maintain suction via the opening 35, and thereby maintains target tissue (or a portion of the target tissue) within the opening 35. In some embodiments, the closed distal end 90 may have a curved end to allow easy insertion of the medical device inside the patient's body, and may also have smooth surface to avoid any accidental damage to the internal parts of the body. Further, the closed distal end 90 may be substantially arcuate, but other shapes such as, spherical, hemi-spherical, stream-lined, or the like may also be contemplated.

The opening 35 may extend along a length of the outer member 30 and may be in fluid communication with the central lumen of the outer member 30. The size and shape of the opening 35 may be based, at least in part, on the size and/or geometry of the outer member 30. Additionally or alternatively, the size and shape of the opening 35 may be based, at least in part, on the size and/or geometry of the target tissue to be received within the opening 35. Further, the opening 35 may optionally include a sharp edge 60 to facilitate tissue severing, as discussed in greater detail below. In some embodiments, the opening 35 may extend along a plane angled (e.g., nonperpendicular and nonparallel) with respect to the longitudinal axis “L” of the outer member 30, creating a window to assist in cutting tissue as will be discussed in further detail below.

In some embodiments, the opening 35 may be defined by the same material that forms the outer member 30, or a separate insert can be disposed within the opening 35 to define its boundary and/or the sharp edge 60 of the opening 35. For example, if the outer member 30 is formed of a biocompatible polymer, the opening 35 may be defined by the same polymeric material, or can be formed from a biocompatible metallic insert about which the outer member 30 may be formed, such as by injection molding.

The inner member 40 may be hollow and adapted to perform tissue resection. As shown, the inner member 40 may be disposed within the central lumen of the outer member 30 and may be concentrically arranged along the longitudinal axis “L” of the outer member 30. Further, the inner member 40 may include a sharp edge 50 to cut the tissue received within opening 35 into smaller pieces. In some embodiments, the sharp edge 50 may also include abrasive coatings or projections, such as barbs and/or saw teeth, facilitating cutting of the target tissue into smaller pieces. The sharp edge 50 may be straight (e.g., extending substantially perpendicular to the longitudinal axis “L”) or angled (e.g., extending substantially nonperpendicular to the longitudinal axis “L”) to facilitate cutting of the tissue. In some embodiments, the sharp edge 50 may be energized through an appropriate electrical connection(s) (not shown) so as to act similarly to an electro-cautery wire. In such an embodiment, the sharp edge 50, and or any other portion of inner member 40 and/or outer member 30, may be conductive and configured to pass electrical current to cut the tissue into smaller pieces. In such an embodiment, one or more of outer member 30, inner member 40, and sharp edge 50 may be made of a material suitable for conducting RF energy. In embodiments including an insert defining the opening 35, as discussed above, the insert may also be optionally conductive and configured to pass electrical current.

The inner member 40 may be configured to move relative to the outer member 30. In some embodiments, for example, the inner member 40 may be axially moveable along the longitudinal axis “L” relative to the outer member 30, while in other embodiments described below, the inner member 40 may be rotatable about the longitudinal axis “L” relative to the outer member 30. The inner member 40 may comprise a hollow tube having a proximal end and a distal end. The proximal end of the inner member 40 may be operably coupled to the handle 10. In some embodiments, the distal end of the inner member 40 may be open, while in other embodiments, the inner member 40 may be closed. The inner member 40 may be configured to cut the received tissue ball when the inner member 40 is moved or rotated with respect to the outer member 30.

During operation, the inner member 40 may be configured to move between an open position, where the opening 35 may be configured to receive the tissue, and a closed position, where the sharp edge 50 of the inner member 40 may sever the received tissue into smaller pieces. For example, in a first embodiment, proximally moving the inner member 40 may cause the inner member 40 to move towards the open position, whereas distally moving the inner member 40 may cause the distal end of the inner member 40 to move towards the closed position to sever the tissue. In the open position, the inner member 40 may be retracted behind (e.g., proximal of) the opening 35 such that the tissue can be received or drawn into the opening 35. Upon receiving tissue in the opening 35, the inner member 40 may be moved distally toward the closed position such that the sharp edge 50 of the inner member 40 cuts the received tissue into smaller pieces.

The outer and inner members 30, 40 may have a substantially circular cross-sectional shape. Other suitable cross-sectional shapes such as elliptical, oval, polygonal, or irregular may also be used. Further, the outer and inner members 30, 40, respectively, may have a generally uniform cross-sectional dimension. Alternatively, the cross-sectional dimensions may vary along their length.

As depicted, the outer and inner members 30 and 40, respectively, may be connected to a handle 10. The handle 10 may be ergonomically designed to allow the physician to easily hold and control the medical device. Further the handle 10 may include one or more connections to various components such as suction source via connection 80 and a power source via connection 100. Also, the handle 10 may include one or more ports (not shown) in communication with one or more of the central lumens of the inner member 40 and the outer member 30. The one or more ports may be configured to communicate one or more medical devices through the inner member 40 and/or the outer member 30.

In some embodiments, the handle 10 may be a separate, discrete component operably coupled to outer and inner members 30, 40, respectively, via soldering, welding adhesive bonding or other coupling mechanisms well known in the art.

In some embodiments, the handle 10 may include a grip coating (i.e., polymeric grip coating) to facilitate handling. Alternatively, the handle 10 may include a frictional element or otherwise textured surface to aid the physician to grip the medical device. The handle 10 may be constructed from any suitable materials such as metal, polymeric, and/or ceramic materials.

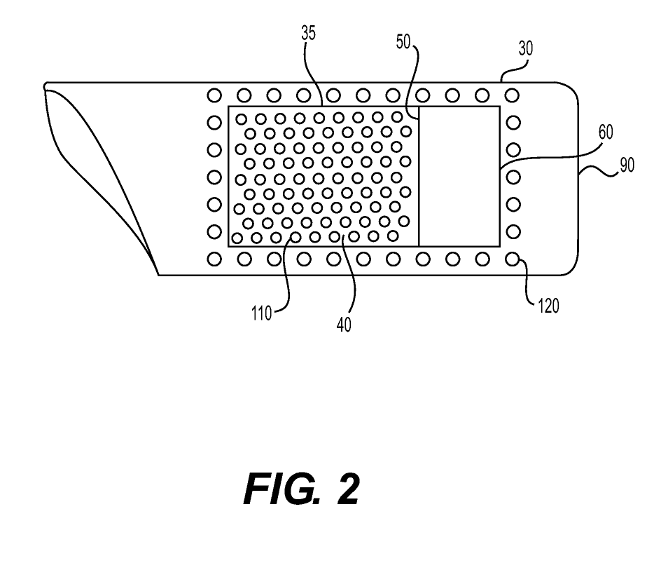

As discussed above, the handle 10 may be coupled to the suction source via connection 80. The suction source, for example, a vacuum pump, may be placed in fluid communication with the outer member 30 and/or the inner member 40. Specifically, the central lumen of the outer and inner members 30, 40, respectively, may be coupled to the suction source via connection 80 and adapted and configured to provide suction through the opening 35. In some embodiments, suction may be applied to draw the tissue excised by the HoLEP procedure into the opening 35 and/or to remove the tissue from the patient's body. Additionally, the suction source may be connected to a suction controller (e.g., a monitoring unit and/or a suction balancer) configured to ensure a proper amount of suction force is applied to the target tissue and to maintain clear passageways, e.g., passageways 110 and 120 (shown in FIG. 2) after severing. Accordingly, the suction source may include a controllable valve that can be opened or closed by the physician to apply a desired amount of suction through the central lumen of the outer member 30. In such embodiments, the valve may be operably controlled via an actuator 70 disposed on the handle 10. In use, for example, the valve may be partially closed so as to reduce the amount of suction applied to the target tissue to reduce trauma. Additionally, if tissue becomes lodged or otherwise stuck (e.g., clogged) in one or more of passageways 110 and 120, described in detail below, the valve may be opened further so as to increase the amount of suction applied and to suck, e.g., dislodge the otherwise stuck tissue from the passageways 110 and/or 120.

In some embodiments, the suction source may be connected to a container or a bag to collect tissue pieces removed from the patient's body. In some embodiments, additional devices such as graspers or clamps may be inserted into the central lumen of the outer member 30 and inner member 40 to remove the tissue from the patient's body.

Actuator 70 may be used for moving the inner member 40 relative to the outer member 30. In some examples, the actuator 70 may include a knob, lever, button, or similar structure for manually moving the inner member 40. Specifically, the actuator 70 may be rigidly connected to the inner member 40 by a mechanical linkage and configured for moving the inner member 40 between the open position and the closed position. The actuator 70 may alternatively be operably coupled to control circuitry and a motor (not shown) which is in turn, mechanically coupled to the inner member 40 to urge or retract inner member 40 relative to outer member 30. For example, a proximal end the inner member 40 may include an actuation carriage (not shown) coupled with the actuator 70. In such instances, actuating the actuator 70 in a first direction may cause the actuation carriage, and consequently the inner member 40, to move towards the open position. Actuating the actuator 70 in a second direction may then cause the actuation carriage, and consequently the inner member 40, to move distally towards the closed position. While FIG. 1 depicts a single actuator 70, it is understood that the handle 10 may include any number of actuators 70 so as to perform the desired functions. For example, the handle 10 may include an additional actuator 70 to control the application of suction from the suction source via connection 80.

A power source may be coupled to one end of the handle 10 via connection 100 to provide power to the medical device 20. In some examples, the power may be required to switch on or switch off the suction source. In other examples, the power may be needed to move the inner member 40 with respect to the outer member 30 for tissue resection. For example, in some embodiments, movement of the inner member 40 relative to the outer member 30 may be facilitated by a motor coupled to an actuation carriage. In such a case, the motor may be powered by a power source via connection 100.

The outer and inner members 30, 40, respectively, may be comprised of any suitable material. For example, the outer and inner members 30, 40, respectively, can be made of the same or similar material. The outer and inner members 30, 40, respectively, may be made so as to have desired flexibility or stiffness along the length of their length. In some embodiments, the outer and inner member 30, 40, respectively may have circular cross-section. Alternatively, other suitable cross-sections such as, but not limited to, rectangular, triangular, oval, irregular, or the like may also be contemplated. The cross-sectional configuration of these members 30, 40 may be uniform or may vary along its length. Additionally, the outer and inner members 30, 40, respectively, may include one or more suitable biocompatible materials, including rigid, flexible, and/or semi-rigid materials. Exemplary materials include, but are not limited to, polymers, metals and metal alloys, and metal-polymer composites.

The introduction sheath and the medical device 20 may optionally include one or more coatings. For example, a suitable low-friction material, such as TEFLON®, polyetheretherketone (PEEK), polyimide, nylon, polyethylene, and/or other lubricious polymer coatings may be applied to one or more components of the medical device 20. For example, one or more lubricious coatings may be applied to facilitate insertion of the introduction sheath and/or medical device into the body and to facilitate movement of inner member 40 relative to outer member 30.

During operation, tissue can be received within the opening 35 by positioning the inner member 40 adjacent target tissue. The received tissue may then be cut by manipulating the inner member 40 in a longitudinal direction along the longitudinal axis “L” with respect to the outer member 30. The resultant cut or resected tissue may then be removed from within the patient's body by any appropriate means, for example, by the use of suction and/or grasping tools.

FIG. 2 illustrates a portion of the elongate member 20 having an outer member 30 and an inner member 40. As shown, the outer member 30 may include a plurality of outer member suction passageways 120 and the inner member 40 may include a plurality of inner member suction passageways 110. The suction passageways 110, 120 may be configured to provide sufficient and constant suction to the medical device such that the target tissue may remain proximate the inner member 40 and outer member 30, thereby preventing the target tissue from bouncing away from the medical device once the inner member 40 is moved toward its closed position. As shown, the suction passageways 110, 120, may extend at least a portion of the inner member 40 and outer member 30, respectively. For example, the suction passageways 110, 120 may extend partially along the length of the inner member 40 and the outer member 30, respectively. While FIG. 2 depicts both the inner member 40 having suction passageways 110 and the outer member 30 having suction passageways 120, in some embodiments, only one of the inner member 40 and outer member 30 may include suction passageways. For example, in some embodiments, inner member 40 may include suction passageways 110, while outer member 30 is solid (e.g., free from or absent any suction passageways 120). Additionally, while an exemplary number of suction passageways 110 and 120 has been depicted, various embodiments may include a greater or lesser number of suction passageways 110 and 120. For example, in any embodiment in which one or both of the inner member 40 and the outer member 30 include suction passageways, each may include a single one or a plurality of passageways.

The suction passageways 110, 120 may have a predetermined arrangement or pattern, for example, a layered, clustered, matrix, honeycomb, or other arrangement. For example, the suction passageways 110, 120 may be discrete, or interconnected. The suction passageways 110, 120 can have different shapes and sizes. For example, each of the passageways 110, 120 can have a uniform size or alternatively, some of the suction passageways 110, 120 may have varying sizes. Varying the sizes of the suction passageways may be useful to set the suction profile of the outer member 30 or inner member 40 for, by example, causing the passageways at the distal ends of the outer member 30 or inner member 40 to have a larger circumference or shape than the passageways at the respective proximal ends. This may cause the suction effect experienced at the passageways at the proximal and distal ends of the outer member 30 or inner member 40 to remain substantially constant. In some embodiments, the suction passageways 110, 120 may be equidistantly spaced from each other, but other variations may also be contemplated. Additionally, the suction passageways 110, 120 may be defined to have a suitable diameter or other dimension such that they are able to provide constant suction to the medical device such that the target tissue remains proximate the outer member 30 and inner member 40.

In some embodiments, the suction passageways 110, 120 may have generally circular cross-sections, but other cross-sectional shapes such as semi-circular, oval, rectangular, spherical, and/or irregular, may also be contemplated. Further, it is understood that the each of the suction passageways 110 and/or the suction passageways 120 may have shapes or sizes differing from each other. For example, in some embodiments, a first suction passageway 110 may be differently sized and/or shaped than a second suction passageway 110. Additionally, in some embodiments, a first suction passageway 120 may be differently sized and/or shaped than a second suction passageway 120.

In some embodiments, the passageways 110, 120 may be defined by removing the material from the body of the outer member 30 and inner member 40, for example by chemical etching, ablation techniques, or any other technique as known in the art. Other configurations may also be contemplated.

In some embodiments, the passageways 110, 120 may be provided with one-way valves or other flow control devices, such as by application of a silicone or elastomer. Such valves or devices may be used to control suction and prevent tissue from entering into or becoming lodged in the passageways. Moreover, such valves or devices may be used to adjust the suction profile of the passageways, for example to provide larger openings at the distal ends of outer member 30 or inner member 40 in order to ensure that the suction effect experienced at passageways at the distal end of the outer member 30 or inner member 40 is substantially the same as the suction effect experienced at passageways at the proximal end of outer member 30 or inner member 40.

During operation, the inner member 40 may be configured to transition between the open position (e.g., either fully or partially open), where the opening 35 may receive the target tissue, and the closed position, in which the opening 35 is closed. As the inner member 40 is actuated to move from the open position to the closed position, the inner member 40 may sever the received tissue into smaller pieces. When in the closed position, the plurality of passageways 110 and/or 120 provide suction through the medical device to maintain contact with the remainder of the target tissue not severed by the inner member 40. Passageways 110, 120 facilitate continuous suction force on the target tissue ball such that even after closing of the opening 35, the remaining portion of the target tissue ball is prevented from escaping or moving away from (e.g., floating away from) the medical device. Accordingly, a physician or medical professional may perform the morcellation/extraction procedure more quickly since they no longer need to reposition the medical device after each time the inner member 40 is moved towards the closed position. Therefore, the overall time to complete the medical procedure is reduced.

In an alternative embodiment, the inner member 40 may be rotated about the longitudinal axis “L” between the open position and the closed position. In such instances, for example, the inner member 40 may include an opening or window defined through a sidewall of the inner member 40. The opening may be configured similarly to the opening 35 defined in the outer member 30. Further, the sharp edge 50 of the inner member 40 may include a longitudinally extending side face (not shown) of the opening of the inner member. During operation, the inner member 40 may be rotated such that the opening of the inner member 40 may align with the opening 35 to receive tissue in the openings, and once received, the inner member 40 may be rotated to sever the received tissue. Specifically, the opening of the inner member 40 may be rotated out of the alignment with the opening 35 of the outer member 30 and as such, the longitudinally directed sharp edge 50 of the inner member 40 may cut (e.g., slice and/or sever) the tissue into smaller pieces. Alternatively, in some embodiments, the inner member 40 may be configured to have C-shape, U-shape, and/or similar shapes having an opening or channel. These shapes may facilitate opening in the inner member 40 such that upon rotation, the inner member 40 and the outer member 30 may together sever the tissue once the tissue is received.

In embodiments in which inner member 40 is rotated relative to outer member 30, it is understood that the inner member 40 and outer member 30 may each have a substantially circular cross-sectional shape. Further, the outer and inner members 30, 40, respectively, may have a generally uniform cross-sectional dimension. Alternatively, the cross-sectional dimensions may vary along their length. Additionally, Actuator 70 may be used for moving the inner member 40 relative to the outer member 30. In some examples, the actuator 70 may include a knob, lever, button, or similar structure for manually rotating the inner member 40. Specifically, the actuator 70 may be rigidly connected to the inner member 40 by a mechanical linkage and configured for rotate the inner member 40 between the open position and the closed position. The actuator 70 may alternatively be operably coupled to control circuitry and a motor (not shown) which is in turn, mechanically coupled to the inner member 40 to rotate inner member 40 relative to outer member 30. For example, a proximal end the inner member 40 may include an actuation carriage (not shown) coupled with the actuator 70. In such instances, actuating the actuator 70 in a first direction may cause the actuation carriage, and consequently the inner member 40, to rotate towards the open position. Actuating the actuator 70 in a second direction may then cause the actuation carriage, and consequently the inner member 40, to rotate in a second direction towards the closed position. While FIG. 1 depicts a single actuator 70, it is understood that the handle 10 may include any number of actuators 70 so as to perform the desired functions.

The following describes an exemplary method for removing excised tissue from a body according to the present disclosure. The method may be utilized for removing the excised tissue from patient's bladder, however, it is understood that the method may be utilized in other parts of the body. The medical device may be introduced through the urethra of the patient and may be advanced toward the bladder such that the medical device may reach a position proximate to a target tissue. As discussed, above, the medical device may include an inner member 40 having a sharpened edge 50, an outer member 30 having an opening 35 extending through a sidewall thereof, wherein the inner member 40 may be configured to move relative to the outer member 30. Additionally, the medical device may include one or more suction passageways 110, 120 extending through at least one or both of the inner member 40 and outer member 30. Once the medical professional positions the medical device proximate the target tissue, the inner member 40 may be moved (e.g., axially translated and/or rotated) relative to the outer member 30 such that the inner member 40 may move towards the open position. In the open position, the target tissue may be drawn into the opening 35 via application of a suction force in fluid communication with the opening 35. Thereafter, the inner member 40 may be moved (e.g., axially translated and/or rotated) relative to the outer member 30 so as to sever the target tissue drawn in the opening 35. While cutting, the remaining portion of the target tissue may be maintained proximate the inner member 40 and outer member 30 via the passageways 110, 120.

Using the exemplary methods described above, the medical device may be used for extraction of resected tissue such as lesions from a patient's body for treatment or diagnostic purposes. The medical device may be a single-use device which can be discarded after one use or may be used again after sterilization.

Other embodiments of the present disclosure will be apparent to those skilled in the art after consideration of the specification and practice of the embodiments disclosed herein. It is intended that the specification and examples be considered as exemplary only, with a true scope and spirit of the disclosure being indicated by the following claims.

Although the present disclosure is described with reference to prostate (prostate gland) surgery, but for a person skilled in the art, it is understood that the present disclosure may also be used in other forms of minimally invasive surgery or percutaneous therapeutic surgery.

1. A medical device, comprising:
an inner member capable of resecting tissue;
an outer member having a window configured to receive tissue; and
a plurality of suction passageways, the plurality of suction passageways extending through at least one or both of the inner member and outer member and located adjacent the window;
wherein the inner member is configured to move relative to the outer member.

2. The medical device of claim 1, wherein the inner member has a sharp edge.

3. The medical device of claim 1, wherein the inner member and outer member are concentrically arranged along a longitudinal axis.

4. The medical device of claim 3, wherein, upon actuation, the inner member is configured to move distally along the longitudinal axis between a proximal open position and a distal closed position.

5. The medical device of claim 4, wherein in the open position, the window is configured to fluidly couple with the suction source, and wherein in the closed position, the plurality of suction passageways are configured to fluidly couple with the suction source.

6. The medical device of claim 3, where upon actuation, the inner member is configured to rotate about the longitudinal axis between an open position and a closed position.

7. The medical device of claim 6, wherein in the open position, the window is configured to fluidly couple with the suction source, and wherein in the closed position, the plurality of suction passageways are configured to fluidly couple with the suction source.

8. The medical device of claim 1, further including a handle coupled to the inner and outer members, the handle operably coupled to a source of vacuum pressure.

9. The medical device of claim 8, further comprising one or more actuators on the handle, the one or more actuators being configured to control movement of the inner member relative to the outer member.

10. A morcellator device, comprising:
an elongate working member, the working member including:
a first member capable of resecting tissue;
a second member having a window extending through a sidewall thereof, wherein the first member is configured to move relative to the second member; and
a plurality of suction passageways extending through at least one or both of the first member and second member and located adjacent the window;
wherein the plurality of suction passageways are configured to fluidly couple to a suction source.

11. The morcellator device of claim 10, wherein the first member and second member are concentrically arranged along a longitudinal axis.

12. The morcellator device of claim 11, wherein, upon actuation, the first member is configured to move distally along the longitudinal axis between a proximal open position and a distal closed position.

13. The morcellator device of claim 12, wherein in the open position, the window is configured to fluidly couple with the suction source, and wherein in the closed position, the plurality of suction passageways are configured to fluidly couple with the suction source.

14. The morcellator device of claim 10, wherein the first member has a sharpened edge.

15. The morcellator device of claim 14, wherein in the open position, the window is configured to fluidly couple with the suction source, and wherein in the closed position, the plurality of suction passageways are configured to fluidly couple with the suction source.

16. A method of removing tissue from a body of a patient, the method comprising:
inserting a medical device proximate to a target tissue, the medical device including
a first member capable of resecting tissue, a second member having a window extending through a sidewall thereof, wherein the first member is configured to move relative to the second member, and a plurality of suction passageways extending through at least one or both of the first member and second member and located to the window;
applying a source of suction to the medical device so as to draw target tissue into the window;
moving the first member relative to the second member so as to sever the target tissue; and
maintaining the suction through the first and second members via the plurality of suction passageways after moving the first member relative to the second member.

17. The method of claim 16, wherein the first member and second member are concentrically arranged along a longitudinal axis.

18. The method of claim 17, wherein moving the first member relative to the second member includes moving the first member distally along the longitudinal axis between a proximal open position and a distal closed position.

19. The method of claim 17, wherein moving the first member relative to the second member includes rotating the first member about the longitudinal axis between an open position and a closed position.

20. The method of claim 16, wherein in the open position, the window is configured to fluidly couple with the source of suction, and wherein in the closed position, the plurality of suction passageways are configured to fluidly couple with the source of suction.
